(19) United States
(12) Patent Application Publication
(10) Pub. No.: US 2002/0013483 A1
(43) Pub. Date: Jan. 31, 2002

(54) Process for the preparation of dianhydrohexitol bisacylates

(75) Inventors: Leonhard Brader, Fischbachau (DE); Norman Haberle, Munchen (DE)

Correspondence Address: William G. Conger, Brooks & Kushman P.C., 22nd Floor, 1000 Town Center, Southfield, MI, 48075-1351, US

(73) Assignee: Consortium fur elektrochemische Industrie GmbH

(21) Appl. No.: 09/892,433

(22) Filed: Jun. 27, 2001

(30) Foreign Application Priority Data
Jul. 13, 2000 (DE) 100 33 952.2

(51) Int. Cl. C07D 493/04

(52) U.S. Cl. 549/464

(57) ABSTRACT

The invention relates to a process for the preparation of dianhydrohexitol bisacylates of the general formula (2),

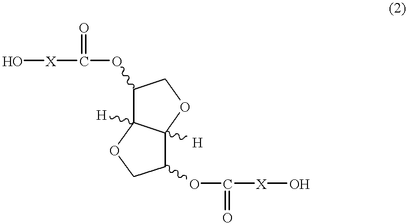

in which free dihydrohexitols are esterified with aromatic, hydroxyl-substituted carboxylic acids of the general formula (3),

HO—CO—X—OH (3)

in a solvent selected from a nonpolar solvent and a mixture of a polar and a nonpolar solvent and in the presence of a highly acidic catalyst, with the removal of water of reaction, where, in the general formulae (2) and (3), X is an optionally fluorine-substituted p-phenylene or 2,6-naphthylene radical.

### BACKGROUND OF THE INVENTION

1. Field of the Invention

The invention relates to a process for the preparation of dianhydrohexitol bisacylates.

2. Background Art

Dianhydrohexitol bisacylates are of considerable importance as precursors for chiral compounds which are able to induce a cholesteric phase in liquid crystals or liquid-crystalline mixtures. Isosorbide bis(4-hydroxy)benzoate is disclosed in EP-A-739 403 as a starting material for compounds whose two hydroxyl groups of the central dianhydrohexitol moiety carry a plurality of sequential aromatic radicals linked by —COO— groups, i.e. substituted benzoyloxybenzoyl esters of anhydrohexitols. The preparation of the isosorbide bis(4-hydroxy)benzoate is not described in EP-A-739 403. A difficulty in the preparation of esters of hydroxybenzoic acid, in particular the esters of 4-hydroxybenzoic acid, is their tendency, especially in the presence of the acidic catalysts frequently used in esterification reactions, to produce considerable proportions of a mixture of hydroxybenzoic acid ester polycondensates of the general formula (1):

HO—(C₆H₄—COO)ₙ—C₆H₄—COOR (1),

in which R is an alkyl or aryl radical, in addition to the desired product. The integer n is greater than 0, and thus depending on the ratio of the reaction components employed, highly condensed polyesters may be formed. The properties of oligoesters and polyesters of this type are frequently problematic; in particular, the isolation of individual species of defined n can only be achieved using complex methods which are commercially uneconomical in large-scale processes. Thus, conventional esterification experiments generally do not yield processable, or especially crystalline compounds, but instead yield resinous mixtures.

This previously described side reaction is unacceptable for the preparation of dianhydrohexitol bis(4-hydroxy) benzoates which are free from more highly condensed esters of the general formula 1 where n is greater than 0, when the preparation of dianhydrohexitol bis(4-hydroxy) benzoates which are acylated on two 4-hydroxyl groups is contemplated. Maintaining a precise and reproducible absorption or reflection wavelength of pigments, possible with well-defined derivatives of dianhydrohexitol acylates, is rendered exceptionally difficult and frequently impossible when mixtures of these compounds, whose helical twisting power (HTP) is subject to great variations owing to the different compositions, is contemplated.

### SUMMARY OF THE INVENTION

The object of the invention was therefore to provide a process by which dianhydrohexitol bisacylates can be obtained in pure form.

### DETAILED DESCRIPTION OF THE PREFERRED EMBODIMENT(S)

The invention relates to a process for the preparation of dianhydrohexitol bisacylates of the general formula (2),

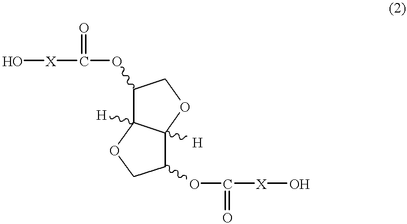

in which the free dihydrohexitols are esterified using aromatic, hydroxyl-substituted carboxylic acids of the general formula (3),

HO—CO—X—OH (3),

in nonpolar solvents or in mixtures of polar and nonpolar solvents, in the presence of a highly acidic catalyst, and with the removal of water of reaction formed, where, in the general formulae (2) and (3), X is an optionally fluorine-substituted p-phenylene or 2,6-naphthylene radical.

The claimed process yields dianhydrohexitol bisacylates of the general formula (2) in a technically simple manner, inexpensively, in high purity and good yields. In particular, few by-products such as hydroxybenzoic acid polycondensates are formed in the process, and their amounts can be reduced to significantly less than 1% by weight. The dianhydrohexitol bisacylates of the general formula (2), which are obtainable in highly pure form, open the pathway to very pure chiral precursors.

In the dianhydrohexitol bisacylates of the general formula (2), the wavy lines denote freely selectable arrangements of the corresponding chemical bonds above or below the drawing plane of the bicyclic dianhydrohexitol system.

Suitable dianhydrohexitol derivatives are, for example, isoiditol (1,4;3,6-dianhydro-L-iditol) and, in particular, isosorbide (=1,4;3,6-dianhydro-D-glucitol) and isomannide (=1,4;3,6-dianhydro-D-mannitol). The carboxylic acid of the general formula (3) employed in the process is, most preferably, 4-hydroxybenzoic acid.

Particularly suitable nonpolar solvents are aliphatic and aromatic hydrocarbons such as hexane, heptane, paraffinic or naphthenic hydrocarbons having a boiling point of from 80 to 160° C. at 0.10 MPa, cyclohexane, toluene, xylenes, trimethylbenzenes such as mesitylene, or pseudocumene. A particularly suitable nonpolar solvent is toluene.

Particularly suitable polar solvents are open-chain ethers such as ethylene glycol dialkyl ethers or diethylene glycol dialkyl ethers, for example diethylene glycol dimethyl ether, cyclic ethers such as dioxane, or esters which are resistant to hydrolysis under the reaction conditions, such as γ-butyrolactone or valerolactone, or dimethylformamide or dimethyl sulfoxide. A particularly suitable polar solvent is diethylene glycol dimethyl ether.

If mixtures of polar and nonpolar solvents are employed, the proportion of polar solvent is from 1 to 99% by weight, preferably at most 50% by weight, in particular at most 20% by weight. A polar solvent is preferably added, since the reaction then proceeds to a greater extent and with a lower proportion of by-products such as those of the general formula (1). Polar and nonpolar solvents can also consist of mixtures of such solvents. The nonpolar and polar solvents and their respective mixtures preferably have a boiling point or boiling range of at most 200° C. at 0.10 MPa.

Particularly suitable acidic catalysts are sulfuric acid, toluenesulfonic acid, benzenesulfonic acid, trifluoroacetic acid and phosphoric acid, and combinations of these acids. The acidic catalyst is preferably sulfuric acid.

The reaction can be carried out at atmosphere pressure (0.1 MPa) or, by use of suitable reactors, for example, autoclaves, at superatmospheric pressure or under reduced pressure. Preferred reaction temperatures extend from 60° C. to 180° C., and are in particular in the range from 105° C. to 120° C.

The free dianhydrohexitol and the carboxylic acid of the general formula (3) are preferably suspended in the solvent at the onset of the reaction.

The reaction proceeds to a particularly great extent if any water introduced with the starting materials and that formed during the reaction is removed in its entirety during the reaction. The water can be removed, for example, by water scavengers, or preferably by azeotropic removal at temperatures of up to 120° C.

It should be noted that all the symbols in the above formulae and all process variables are defined independently of one another. In the following examples, unless stated otherwise, all amounts and percentages are based on the weight, all pressures are 0.10 MPa (abs.) and all temperatures are 20° C.

### EXAMPLE 1

This example illustrates the preparation of isosorbide 2,5-bis(4-hydroxybenzoate).

219.2 g (1.5 mol) of isosorbide, 415 g (3.005 mol) of 4-hydroxybenzoic acid and 12 g of 50% strength sulfuric acid were suspended in a mixture of 800 g of toluene and 35 g of diethylene glycol dimethyl ether, and the mixture heated to 108° C. to 110° C. with stirring. The water introduced with the sulfuric acid and formed during the reaction was removed by azeotropic distillation, and the mixture was then cooled to 85° C., following which 1.8 l of ethyl acetate were added. The mixture was neutralized by slow addition of 250 ml of saturated sodium bicarbonate solution, and the aqueous phase was then separated. The organic phase was washed twice with water, and 1.2 l of ethyl acetate were then removed by distillation. Following cooling with stirring, the resultant crystals were removed by filtration, and when dried, yielded 465 g of the target product, corresponding to a yield of 80.3% of theory. After recrystallization from ethyl acetate, the product had a melting point of 213° C. Evaluation of the NMR spectrum showed a proportion of products of the general formula 1 of significantly less than 1%, which, owing to the small size of the signal, could not be determined more accurately.

### EXAMPLE 2

This example illustrates the preparation of isomannide 2,5-bis(4-hydroxybenzoate).

Isomannide was converted into isomannide 2,5-bis(4-hydroxybenzoate) by the process of Example 1. Removal of water by azeotropic distillation was also carried out in the same way as in Example 1. The target product was obtained in a yield of 57% of theory, and had a melting point of 233° C. The content of oligomers of the general formula 1 is less than 1%.

### EXAMPLE 3

This example illustrates the preparation of isosorbide 2,5-bis(6-hydroxy)-naphthoate-2.

33.1 g (0.24 mol) of isosorbide in 150 ml of toluene containing 4 ml of 50% strength sulfuric acid and 25 ml of diethylene glycol dimethyl ether were added to 92 g (0.49 mol) of 2-hydroxy-6-naphthoic acid, and the mixture was refluxed at 100-112° C. for 15 hours with azeotropic removal of the water of reaction. After work-up of the 30% esterified reaction mixture by addition of 100 ml of ethyl acetate at 20° C. and filtration of the insoluble product followed by washing with 50 ml of ethyl acetate, 32.4 g of crude product were obtained. After recrystallization from glacial acetic acid, 14.5 g of pure product having a melting point of 233-235° C. was obtained. The yield was 12.4% of theory.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention. By the terms “a” and “an” in the claims is meant “at least one” unless specified to the contrary.

What is claimed is:

1. A process for the preparation of dianhydrohexitol bisacylates of the general formula (2), 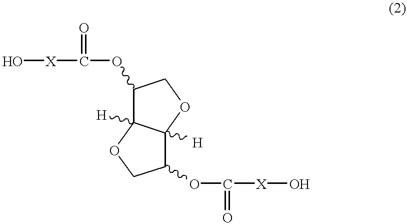
comprising:
esterifying one or more dihydrohexitols with at least one aromatic, hydroxyl-substituted carboxylic acid of the general formula (3),
HO—CO—X—OH (3),
in a nonpolar solvent or a mixture of at least one polar and at least one nonpolar solvent, in the presence of an acidic catalyst, and with removal of water of reaction formed thereby, wherein X is an optionally fluorine-substituted p-phenylene or 2,6-naphthylene radical.

2. The process of claim 1, in which the nonpolar solvent employed is toluene.

3. The process of claim 1, in which a mixture of a polar and a nonpolar solvent is employed.

4. The process of claim 2, in which a mixture of a polar and a nonpolar solvent is employed.

5. The process of claim 1, wherein the polar solvent employed comprises diethylene glycol dimethyl ether.

6. The process of claim 2, wherein the polar solvent employed comprises diethylene glycol dimethyl ether.

7. The process of claim 3, wherein the polar solvent employed comprises diethylene glycol dimethyl ether.

8. The process of claim 4, wherein the polar solvent employed comprises diethylene glycol dimethyl ether.

9. The process of claim 1, wherein the catalyst employed comprises sulfuric acid.

10. The process of claim 2, wherein the catalyst employed comprises sulfuric acid.

11. The process of claim 3, wherein the catalyst employed comprises sulfuric acid.

12. The process of claim 5, wherein the catalyst employed comprises sulfuric acid.

13. The process of claim 1, wherein the carboxylic acid of the general formula (3) comprises 4-hydroxybenzoic acid.

14. The process of claim 2, wherein the carboxylic acid of the general formula (3) comprises 4-hydroxybenzoic acid.

15. The process of claim 3, wherein the carboxylic acid of the general formula (3) comprises 4-hydroxybenzoic acid.

16. The process of claim 5, wherein the carboxylic acid of the general formula (3) comprises 4-hydroxybenzoic acid.

17. The process of claim 9, wherein the carboxylic acid of the general formula (3) comprises 4-hydroxybenzoic acid.
